(19) United States
(12) Patent Application Publication
(10) Pub. No.: US 2007/0055177 A1
(43) Pub. Date: Mar. 8, 2007

(54) CAPSULE CONTAINER

(75) Inventors: Masaaki FUKUDA, Tokyo (JP); Masaaki NAKASHIMA, Tokyo (JP); Taro NAKASENDO, Tokyo (JP)

Correspondence Address: GREENBLUM & BERNSTEIN, P.L.C., 1950 ROLAND CLARKE PLACE, RESTON, VA, 20191, US

(73) Assignee: PENTAX CORPORATION, Tokyo (JP)

(21) Appl. No.: 11/467,615

(22) Filed: Aug. 28, 2006

(30) Foreign Application Priority Data
Sep. 7, 2005 (JP) P2005-258884

(51) Int. Cl. A61B 5/103

(52) U.S. Cl. 600/593

(57) ABSTRACT

A capsule container, for holding a used capsule, includes a body and a cover. The body has a holder to hold the capsule, and the cover is attached to the body. When the capsule is held by the holder, the cover is attached to the body, so that the capsule and the holder are sealed by the covers.

### BACKGROUND OF THE INVENTION

1. Field of the Invention

The present invention relates to a capsule recovery tool, especially to a capsule recovery tool for recovering a capsule-type medical instrument, such as a used capsule endoscope.

2. Description of the Related Art

Recently, a capsule endoscope device for observing inside a digestive organ has been developed. In a capsule endoscope device, a capsule, for photographing inside a body, is swallowed by a subject person, and images of the inside the body of the subject person are generated by processing image data obtained by the capsule.

In a capsule endoscope device, a capsule that is excreted from a body after photographing the inside thereof is recovered from excrement. The recovered capsule and a capsule recovery tool should be sealed when they are brought, because excrement may be attached to them. Therefore, a capsule recovery device, including a bag for sealing a recovered capsule and a capsule recovery tool, is known.

As mentioned above, in a case where a capsule-type medical instrument (hereinafter, simply called a capsule) and a capsule recovery tool are held in a capsule holder, usually, a bag and so on are required. Further, to seal a used capsule and a capsule recovery tool, the periphery of the mouth of the bag should be processed. On the other hand, manipulation of some types of capsule recovery tools such as rod type tools, which are not suitable for being held or being brought as they are, sometimes prevent a prompt operation, because they need to be bent or folded when they are held.

### SUMMARY OF THE INVENTION

Therefore, an objective of the present invention is to provide a capsule container in which a used capsule is sealed with an easy operation.

A capsule container for holding a used capsule includes a body and a cover. The body has a holder to hold the capsule, and the cover is attached to the body. When the capsule is held by the holder, the cover is attached to the body, so that the capsule and the holder are sealed by the covers.

A pair of the holders whose distance in between is the same as the diameter of the capsule can be provided, and the capsule can be put between the holders. A pair of the holders may have a corrugated shape.

The holder may rotate about an axis. The body may have a tubular shape, and the axis may run in a perpendicular direction to a direction in which the body extends. The cover may have a tubular shape whose diameter is shorter than the length of the capsule container, and the holder holding the capsule may be rotated, so that the cover may be attached to the body.

The cover may be detachably attached to the body. The holder may have a half-cylindrical shape. The body may have a handle for a user to hold the body.

### BRIEF DESCRIPTION OF THE DRAWINGS

The present invention will be better understood from the description of the preferred embodiments of the invention set forth below, together with the accompanying drawings, in which:

FIG. 1 is a front view representing a capsule container of a first embodiment with a capsule that is not held;

FIG. 2 is a side view representing the capsule container of the first embodiment with the capsule that is not held;

FIG. 3 is a bottom view representing a body of the capsule container of the first embodiment when the capsule is not held;

FIG. 4 is a front view representing the capsule container is of the first embodiment when the capsule is sealed;

FIG. 5 is a side view representing the capsule container of the first embodiment when the capsule is sealed;

FIG. 6 is a bottom view representing the body of the capsule container of the first embodiment when the capsule is sealed;

FIG. 7 is a front view representing a capsule container of a second embodiment with a capsule that is not held;

FIG. 8 is a side view representing the capsule container of the second embodiment with the capsule that is not held;

FIG. 9 is a front view representing the capsule container of the second embodiment with a capsule that is held; and

FIG. 10 is a front view representing the capsule container of the second embodiment when the capsule is sealed.

### DESCRIPTION OF THE PREFERRED EMBODIMENTS

Hereinafter, the first embodiment of the present invention is described with reference to the attached drawings.

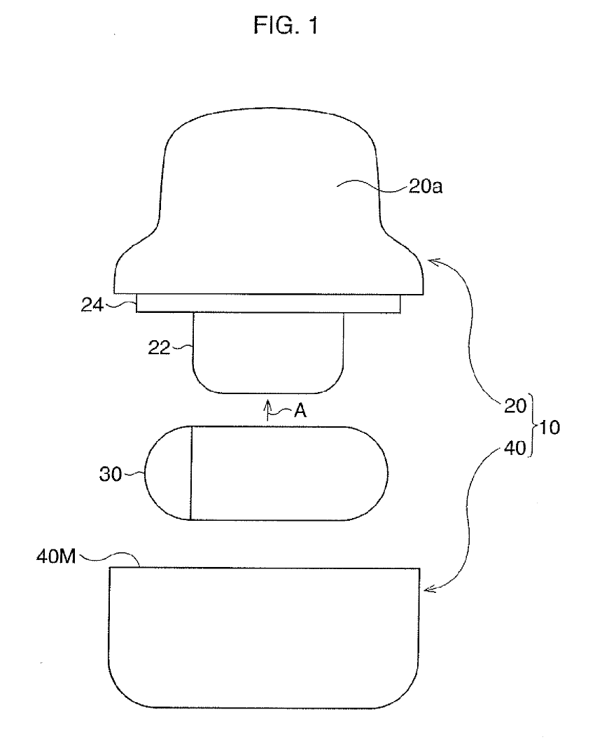

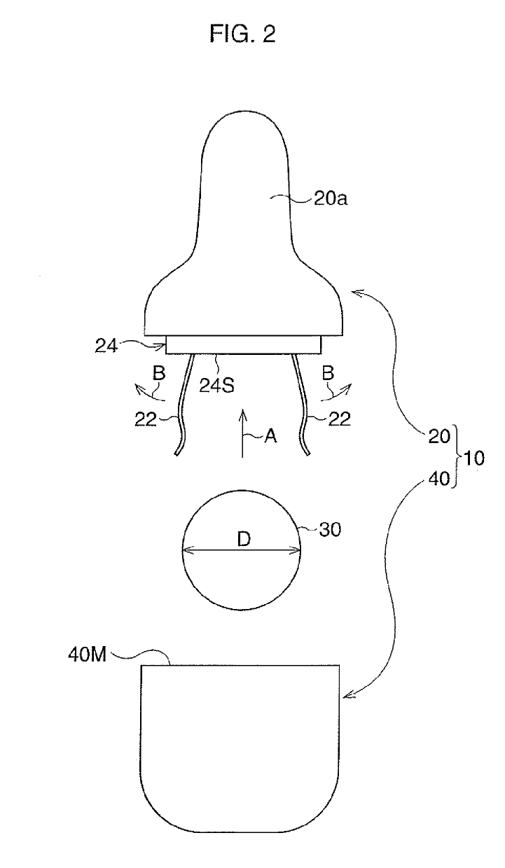

As shown in FIGS. 1 to 3, a capsule container 10 includes a body 20, and a cover 40 that is detachably attached to the body 20. The cover 40 is detached from the body 20 for recovering a capsule endoscope (hereinafter, simply called a capsule) 30, as shown in FIGS. 1 and 2. Then, the cover 40 is attached to the body 20 before and after recovering the capsule 30. In the body 20, a pair of holders 22 are provided (see FIG. 2). The capsule 30 is captured and held by the holders 22 by an operation of a user as represented by the arrow A. To make such an operation easy, a handle 20a whose width is smaller than other parts of the body 20 is provided A user grabs the handle 20a with his or her fingers to hold the body 20, and presses the capsule 30 into the holders 22, to capture the capsule 30 as represented by the arrow A.

In the body 20, a projected part 24 is provided. The cover 40 can remain attached to the body 20, using the projected part 24 that is fit into a mouth 40M of the cover 40. Between the projected part 24 and the mouth 40M, there exists suitable friction, so that the operations for attaching the cover 40 to the body 20 and for detaching the cover 40 from the body 20 are easy.

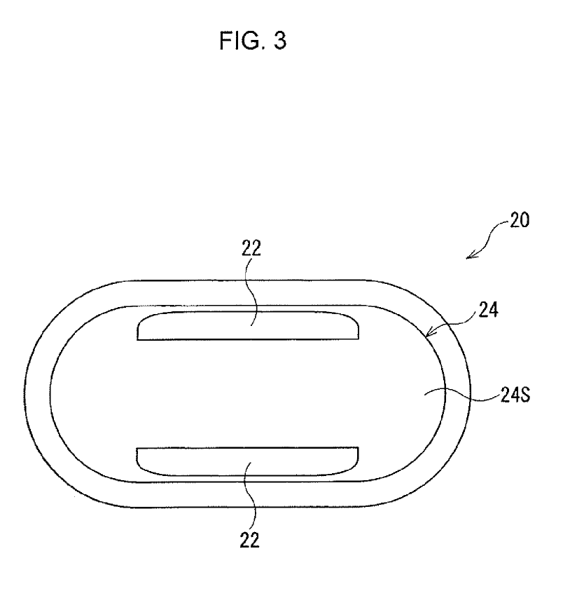

The pair of holders 22 are provided on a surface 24S of the projected part 24, and the distance between the pair of holders 22 is almost the same as the diameter D of the capsule 30 (see FIGS. 2 and 3). Therefore, the capsule 30 can be put between the pair of holders 22. The holders 22 are slightly corrugated in a direction parallel to the projected part surface 24S, although they extend almost perpendicularly from the projected part surface 24S. Further, the holders 22 can expand and contract suitably in the direction represented by the arrow B; therefore, the capsule 30 is easily captured and held tightly by the holders 22.

Note that the body 20 and the cover 40 are both made of plastic. The size and shape of the holders 22 can be adjusted, and the holders 22 are formed by plastic having suitable elasticity.

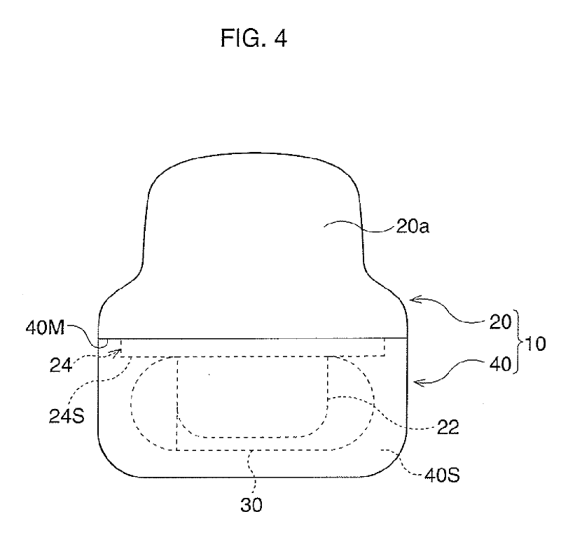

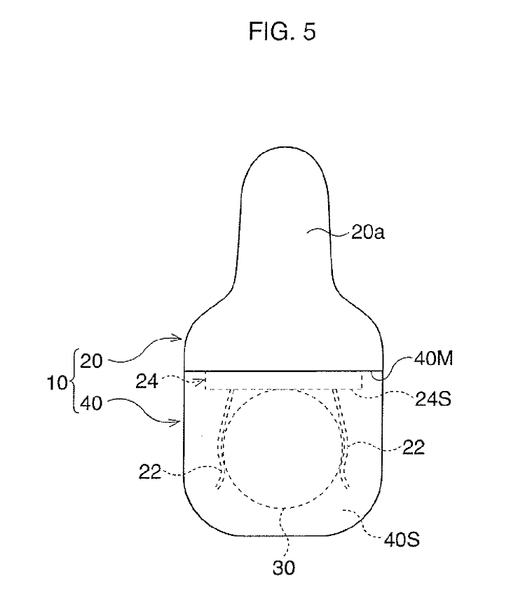

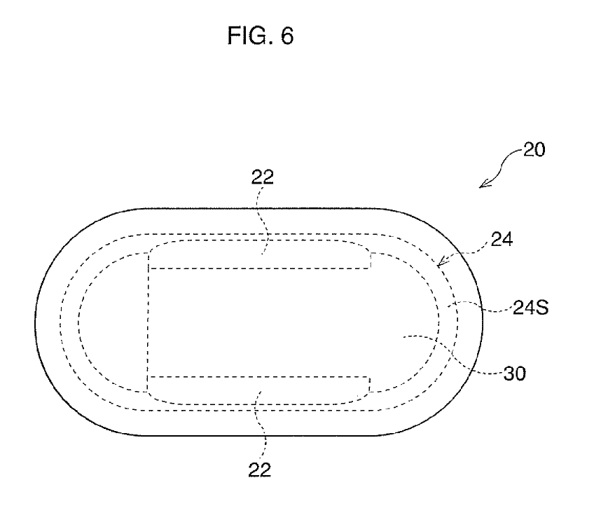

When the capsule 30 is held by the pair of holders 22 and the cover 40 is attached to the body 20, the pair of the holders 22 and the capsule 30 are sealed in a closing space 40S of the cover 40 (see FIGS. 4 and 5). The projected part 24 is designed so that the projected part surface 24S and the held capsule 30 almost overlap; that is, the outline of the capsule 30, which is held by the holders 22, and the outline of the projected part surface 24S almost overlap when they are seen from a direction perpendicular to the projected part surface 24S (see FIG. 6).

As it is clear from the above explanation, the capsule container 10 is designed so that the closing space 40S of the cover 40 is slightly larger than the capsule 30, in such a way that the capsule container 10 is miniaturized. Note that in the aforementioned figures, the projected part surface 24S is represented to be clearly smaller than the mouth 40M of the cover 40, and the capsule 30 is represented to be clearly smaller than the closing space 40S of the cover 40 for convenience of explanation; however, in practice, the projected part surface 24S is slightly smaller than the mouth 40M of the cover 40, and the closed capsule 30 occupies almost all area of the closing space 40S.

In the first embodiment, as explained above, the capsule 30 and the holders 22, to which excrement may be attached after excretion from the body, can be sealed inside the capsule container 10 to assist in recovering the capsule 30. Further, the capsule container 10 is miniaturized, so that carrying a pair of the capsule 30 and the capsule container 10 is easy, both before and after using the capsule 30. The capsule 30 can be recovered and covered by a simple operation; that is, by holding the body 20 and capturing the capsule 30 using the holders 22.

Hereinafter, a second embodiment of the present invention is explained, mainly focusing on the differences from the first embodiment. Note that in the following figures for the second embodiment, the identical components as those in the first embodiment have identical reference numerals.

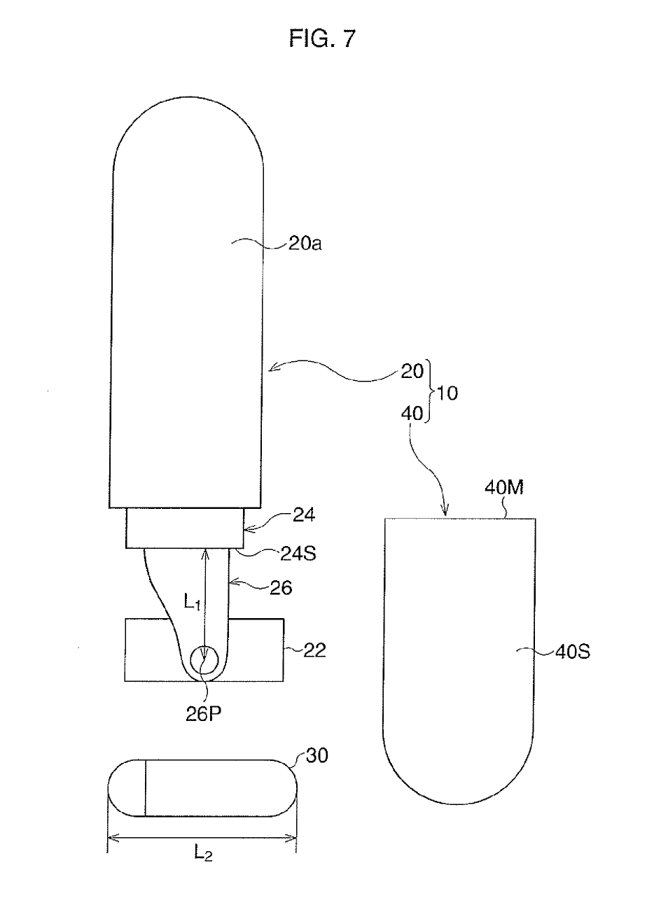

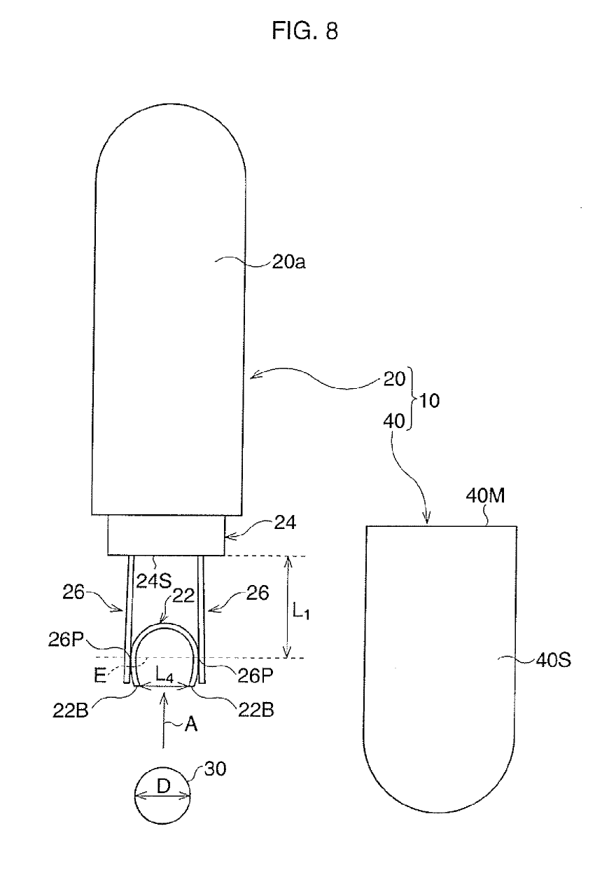

In the second embodiment, both the body 20 and the cover 40 have a long tubular shape (see FIGS. 7 to 9). When the cover 40 is attached to the body 20, the capsule container 10 has an almost cylindrical shape. The holder 22 is a simple member that has a half-cylindrical shape (see FIG. 8), and a pair of supporters 26 that extend from the projected part surface 24S almost perpendicularly, and that support the holder 22, are provided in the body 20.

The holder 22 is rotatable around the axis E connecting two contact points 26P, which are points contacting the holder 22 on each of the pair of supporters 26. That is, the axis E runs in a perpendicular direction to the direction in which the body 20 extends. The distance L₁ between the projected part surface 24S and the contact points 26P is longer than half of the length L₂ of the capsule 30, so that the holder 22 holding the capsule 30 is rotatable over 360 degrees (see FIG. 7).

The distance L₄ between the two lower ends 22B of the holder 22 is shorter than the diameter D of the capsule 30, although the distance between the two contact points 26P is almost the same as the diameter D of the capsule 30 (see FIG. 8). Therefore, the capsule 30, captured in the direction represented by the arrow A by an operation of a user, is tightly held by the holder 22.

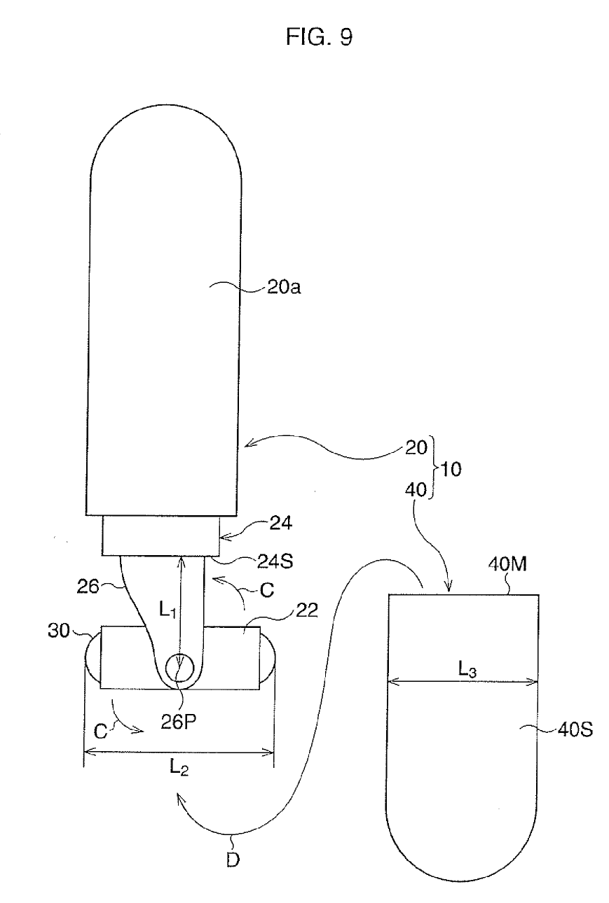

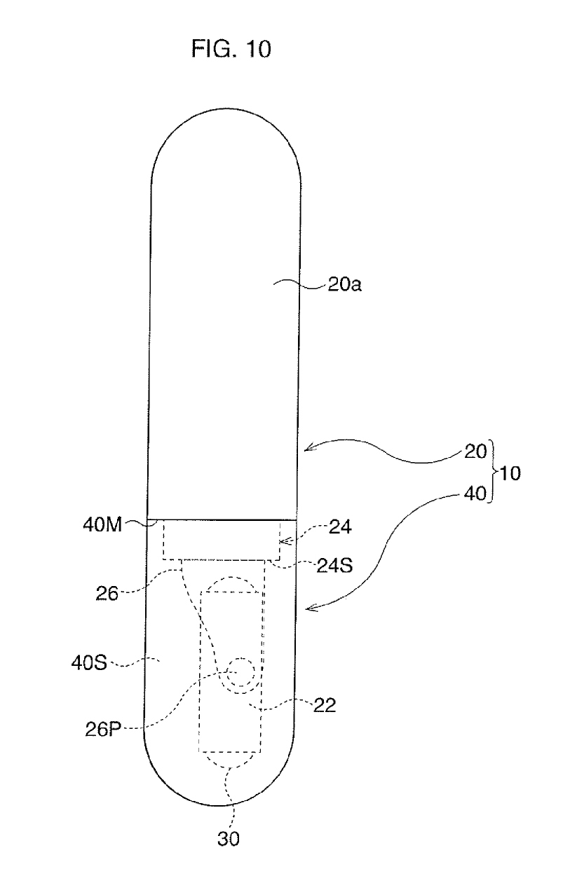

Because the length L₂ of the capsule 30 is longer than the width L₃ of the tubular cover 40, when the capsule 30 is just held by the holder 22 as shown in FIG. 9 (that is, when the longitudinal direction of the holder 22 is almost perpendicular to the direction in which the body 20 extends), the cover 40 can not be attached to the body 20. Then, the holder 22 holding the capsule 30 is rotated until the capsule 30 is almost parallel to the body 20, as represented by the arrow C. As a result of this rotation, the cover 40 becomes attachable to the body 20, as represented by the arrow D, and the capsule 30, holder 22, and supporter 26 can all be sealed inside the closing space 40S of the cover 40 (see FIG. 10).

Note that the length of the body 20 (that is, the length between the upper and lower ends of the body 20 in the vertical direction in FIGS. 7 to 9), is preferably short in order not to disturb the field of vision of a user who is operating the capsule container 10 for capturing the capsule 30. Therefore, for example, in terms of proportion, the length of the body 20 is designed to be less than 5 centimeters when the capsule container 10 targets a capsule 30 having a length of about 2 to 3 centimeters.

In the second embodiment, as explained above, the capsule container 10 can be further miniaturized, where the width L₃ of the tubular cover 40 (that is, the width of the capsule container 10) is shorter than the length L₂ of the capsule 30, by providing the rotatable holder 22.

Note that the shapes and materials of the body 20, the cover 40, and other elements included in the capsule container 10 are not limited to those of the first and second embodiments, so long as the capsule container 10 can recover the capsule 30 to be sealed. For example, the capsule container 10 may have shapes other than a cylindrical shape, and the body 20 may have recesses for easy holding by a user who is capturing the capsule 30.

Further, in the above embodiments, the capsule container 10 is used for recovering the capsule endoscope 30; however, the capsule container 10 may be used for other purposes. For example, the capsule container 10 may be used for recovering a capsule medical device that collects cells in a required part of a body or that sends medicine inside a patient body, as by radio remote control.

This invention is not limited to that described in the preferred embodiments; namely, various improvements and changes may be made to the present invention without departing from the spirit and scope thereof.

The present disclosure relates to subject matter contained in Japanese Patent Application No. 2005-258884 (filed on Sep. 7, 2005), which is expressly incorporated herein, by reference, in its entirety.

1. A capsule container for holding a used capsule, comprising:
a body that has a holder to hold said capsule; and
a cover that is attached to said body;
wherein when said capsule is held by said holder, said cover is attached to said body, so that said capsule and said holder are sealed by said cover.

2. The capsule container according to claim 1, wherein a pair of said holders whose distance is the same as the diameter of said capsule is provided, and said capsule is put between said holders.

3. The capsule container according to claim 1, wherein a pair of said holders has a corrugated shape.

4. The capsule container according to claim 1, wherein said holder can rotate about an axis.

5. The capsule container according to claim 4, wherein said body has a tubular shape, and said axis runs in a perpendicular direction to a direction in which said body extends.

6. The capsule container according to claim 4, wherein said cover has a tubular shape whose diameter is shorter than the length of said capsule container, and said holder holding said capsule is rotated, so that said cover can be attached to said body.

7. The capsule container according to claim 1, wherein said cover is detachably attached to said body.

8. The capsule container according to claim 1, wherein said holder has a half-cylindrical shape.

9. The capsule container according to claim 1, wherein said body has a handle for a user to hold said body.
